(19) United States
(12) Patent Application Publication
(10) Pub. No.: US 2003/0181358 A1
(43) Pub. Date: Sep. 25, 2003

(54) Use a high-molecular-weight extracellular haemoglobin as a blood substitute

(76) Inventors: Franck Zal, Poujean-Morlais (FR); Andre Toulmond, Paris (FR); Francois Lallier, Saint-Pol de Leon (FR)

Correspondence Address: YOUNG & THOMPSON, 745 SOUTH 23RD STREET 2ND FLOOR, ARLINGTON, VA, 22202

(21) Appl. No.: 10/296,982

(22) PCT Filed: May 17, 2001
(86) PCT No.: PCT/FR01/01505

(30) Foreign Application Priority Data
May 31, 2000 (FR) 00/07031

(51) Int. Cl. A61K 38/42; C07K 14/805

(52) U.S. Cl. 514/6; 530/385

(57) ABSTRACT

The invention concerns the use as blood substitute of a high-molecular-weight extracellular haemoglobin of about 3 to about 4 million daltons, comprising polymerised globin chains, containing free cysteines capable of binding to NO and/or SNO groups and whereof the P₅₀ is about 6 to 7 mm Hg at 37° C.

The invention concerns the use of a high molecular weight extracellular haemoglobin as a blood substitute.

The invention also concerns new blood substitutes, including a high molecular weight extracellular haemoglobin.

Blood is a complex liquid, whose main function is to transport oxygen and carbon dioxide, to ensure the respiratory processes. This function is performed by the haemoglobin molecule, which is found in the red blood corpuscles.

In mammals the haemoglobin molecule is made up of four similar functional polypeptide chains in pairs (2 α-type globin chains and 2 β-type globin chains). Each of these polypeptide chains possesses the same tertiary structure of a myoglobin molecule (11).

Haem, the active site of haemoglobin, is a tetrapyrrole protoporphyrin ring, containing a single iron atom at its centre. The iron atom, which fixes oxygen, contracts 6 coordinate bonds: four with the nitrogen atoms in the porphyrin, one with the F8 proximal histidine and one with the oxygen molecule during oxygenation of the globin.

There are currently problems with the supply of blood, as the number of donors is falling due to the fear of contamination. The last few years have therefore seen an acceleration in research into blood substitutes. Attempts are being made to design artificial blood substitutes capable of eliminating the risk of transmitting infectious diseases, which would also bring freedom from problems of blood group compatibility.

Up till now, research has chiefly been concerned on the one hand with the synthesis of chemicals (23) and on the other hand with the synthesis of biological products (24,25).

With regard to the first area of research, use has been made of perfluorocarbons (PFCs). PFCs are chemicals capable of transporting oxygen, and able to dissolve a large quantity of gas, such as oxygen and carbon dioxide.

Efforts are currently being made to produce emulsions of these products which could be dispersed in the blood more efficiently (29-31).

The advantage of PFCs lies in their oxyphoric capacity which is in direct proportion to the quantity of oxygen in the lungs. Moreover, due to the fact that there is no membrane to cross, PFCs can transport oxygen to tissues more rapidly. However, the long-term effects of the retention of these products in the organism is not known. When these products were used for the first time during the 1960s, as a blood substitute in mice (23,28,32), the side effects were very considerable. The PFCs were not satisfactorily eliminated from the circulation and accumulated in the tissues of the organism, causing oedemas.

In the 1980s, a new version of PFC was tested in the clinical phase. But problems of storage, financial cost, considerable side effects and the low efficiency of this compound prevented the extension of its marketing (33,34,35).

Recently, a new generation of PFC's has been developed (PFBO perfluorooctylbromide). A new product (29) is undergoing clinical trials in the USA, but it has already been found that an increase in the quantity of oxygen in the blood can give rise to an accumulation of oxygen in the tissues, which is dangerous for the organism (formation of superoxide-type radical oxygen).

Thus, in spite of the progress being achieved, the side effects of these compounds are still too considerable to allow marketing on a large scale.

As regards the second area of research, work has been carried out on the development of blood substitutes by modifying the structure of natural haemoglobin 24,36). To obtain a modified-haemoglobin-type blood substitute, use is made of haemoglobins from genetically modified microorganisms, or of human or animal origin, in particular the bovine haemoglobin molecule. Bovine haemoglobulin does differ slightly from human haemoglobin as regards immunology, but it transports oxygen to the tissues more easily. Nevertheless, the risk of viral or spongiform-encephalopathy-type contamination still remains considerable.

To be functional, the haemoglobin must be in contact with an allosteric effector, 2,3-diphosphoglycerate (2,3-DPG), present only inside the red corpuscles (38). Moreover, without 2,3-DPG and other elements present in the red corpuscles, such as methemoglobin reductase, haemoglobin undergoes a self-oxidation process and loses its capacity to transport oxygen or carbon dioxide.

These processes can be eliminated by modifying the structure of the haemoglobin, and more precisely by stabilising the weak bonds of the tetrameric molecule between the two α and β dimers (39). A number of modifications have been tested: covalent bond between two α chains, between two β chains or between α and β (40,41).

Attempts have also been made to polymerise the tetrameric molecules or to conjugate them with a polymer known as polyethylene glygol (PEG) (42). These modifications result in stabilisation of the molecule and an increase in its size, preventing its elimination by the kidneys.

Annelids have been extensively studied for their extracellular haemoglobin (10,44). These extracellular haemoglobin molecules are present in the three classes of Annelids: Polychaetes, Oligochaetes and Achaetes and even in the Vestimentifers. These are giant biopolymers, made up of approximately 200 polypeptide chains belonging to 6 or 7 different types, which are generally grouped together in two categories. The first category, consisting of 144 to 192 elements, groups together the “functional” polypeptide chains, carrying an active site and capable of reversibly binding oxygen; these are globin-type chains of masses between 15 and 18 kDa, which are very similar to the α- and β-type chains of vertebrates. The second category, consisting of 36 to 42 elements, groups together “structural” polypeptide chains having few or no active sites but allowing the assembling of the “twelfths”.

The first images obtained of extracellular haemoglobins of Arenicola (45,46) have revealed hexagonal elements. Each haemoglobin molecule is made up of two superimposed hexagons (47,48), called a hexagonal bilayer, and each hexagon is itself made up of six elements in the form of a drop of water (49,50) called a hollow globular structure (51,54) or “twelfth”. The native molecule is formed from twelve of these sub-units, of a molecular mass of approximately 250 kDa.

There is particular interest in Arenicola marina, a polychaete annelid of the intertidal ecosystem. Moreover, the structure of its extracellular haemoglobin is already known (60).

Studies have already been carried out of the use of the extracellular haemoglobin of the nightcrawler (Lumbricus terrestris) as a blood substitute (2). However, this haemoglobin would not be suitable, firstly due to probable disturbance of the vasodilation and/or vasoconstriction of blood vessels due to the absence of free cysteine residues (71) and, secondly, this haemoglobin presents too weak an affinity with oxygen, i.e. a high P₅₀.

Up to now, none of the available blood substitutes makes it possible to avoid the problems of contamination and blood-group compatibility, even though they have no side effects.

The invention makes it possible to remedy these disadvantages.

The object of the invention is to propose new blood substitutes making it possible to eliminate problems due to lack of donors.

A subject of the invention is also to propose new blood substitutes making it possible to avoid the problems of transmissions of infectious diseases during blood donation.

The invention also relates to new blood substitutes making it possible to preserve organs during transplantations.

The invention also relates to new blood substitutes allowing freedom from problems of blood-group compatibility, in particular during transfusions.

The invention concerns the use, as a blood substitute, of an extracellular haemoglobin having a molecular weight of approximately 3 to approximately 4 million daltons, comprising chains of polymerised globins, containing free cysteines capable of binding to NO and/or SNO groups, and having a P₅₀ of approximately 6 to approximately 7 mm Hg at 37° C.

The invention also concerns a blood substitute, in particular a human blood substitute, comprising an extracellular haemoglobin having a molecular weight of approximately 3 to approximately 4 million daltons, comprising chains of polymerised globins, containing free cysteines capable of binding to NO and/or SNO groups, and having a P₅₀ of approximately 6 to approximately 7 mm Hg at 37° C.

The term “blood substitute” defines a biological product capable of replacing the haemoglobin present in the red blood corpuscles and capable of performing its functions as a transporter of gas (oxygen and carbon-dioxide). This blood substitute also has to supply oxygen to the tissues, where it becomes charged with CO₂, to release this gas at the exchange surfaces (lungs).

The term “extracellular haemoglobin” refers to a haemoglobin not contained in the cells and dissolved in the blood.

The term “chains of polymerised globins” defines covalent associations of globin chains.

The number of free cysteins capable of binding to NO and/or SNO groups can range from approximately 120 to approximately 150, and in particular approximately 120 to approximately 130.

An example of a test making it possible to determine binding to NO groups is that used by Jia et al. (71).

An example of a test making it possible to determine binding to SNO groups is that used by Jia et al. (71).

P₅₀ is a parameter used to measure the affinity of a respiratory pigment to oxygen, which corresponds to 50% oxygen saturation of the binding sites of a respiratory pigment.

This corresponds to oxygen's efficiency in fixing to haem.

The P₅₀ can be measured using the hemox technique (1).

According to an advantageous embodiment, in the blood substitute of the invention, the extracellular haemoglobin cooperativity coefficient is 2 to 3 (n₅₀).

The haemoglobin cooperativity coefficient (n₅₀) is defined as being the parameter used to estimate the oxygen-binding capacity of the different active sites of the globin chains.

The n₅₀ can be measured on the oxygen saturation curves of a respiratory pigment, obtained using the hemox technique.

According to an advantageous embodiment, in the blood substitute of the invention, the globin chains of extracellular haemoglobin are stabilised between themselves, by covalent bonds, in particular intermolecular disulphide bridges, and the globin chains are auto-stabilised by intramolecular disulphide bridges.

The expression “the globin chains of extracellular haemoglobin are stabilised between themselves, by covalent bonds” refers to the presence of interchain disulphide bonds between two or more globin chains.

The expression “the globin chains are auto-stabilized” refers to the presence of intrachain disulphide bonds on each globin chain.

According to an advantageous embodiment, in the blood substitute of the invention, the extracellular haemoglobin comprises structural chains conferring a hexagonal structure on the haemoglobin.

The term “structural chains” designates polypeptide chains having little or no haem, which maintain the hexagonal structure of the molecule.

According to an advantageous embodiment, in the blood substitute of the invention, the extracellular haemoglobin is capable of neutralising toxic compounds, such as hydrogen sulphide.

The expression “the extracellular haemoglobin is capable of neutralising toxic compounds” refers to the fixation of hydrogen sulphide on free cysteine residues making it possible to reduce, or even eliminate, this compound from the internal environment of an organism. Once fixed, the hydrogen sulphide becomes non-toxic.

The term “toxic compounds” defines for example a chemical or biological element which will give rise to physiological disturbances or pathological disorders in an organism.

An example of a test to verify the neutralisation of toxic compounds is that used in the two publications (59,74), a test involving dosage by chromatography in the gaseous phase.

According to an advantageous embodiment, in the blood substitute of the invention, the extracellular haemoglobin does not necessitate any cofactor to liberate any oxygen possibly fixed on the haemoglobin.

The expression “the extracellular haemoglobin does not necessitate any cofactor” refers to a haemoglobin dissolved in the blood, which is capable of releasing its oxygen without the involvement of another molecule, as is the case for intracellular haemoglobines which involve, for example, 2,3-DPG.

The haemoglobin of vertebrates is contained in a nucleated cells or red corpuscles. Inside these cells, the main cofactor found is 2,3-DPG which enables fixed oxygen to be released.

If the 2,3-DPG were found in the presence of extracellular haemoglobin, this would have no effect on the release of oxygen by this pigment.

According to an advantageous embodiment, in the blood substitute of the invention, the extracelluar haemoglobin possesses the following properties:

it is non-toxic

it has no pathogenic agent

it keeps for at least 6 weeks at 4° C. without oxidation

it is transfusable into all blood types

it has a sufficiently long residence time to ensure regeneration into natural haemoglobin of the organism into which it is transfused

it is eliminated by the organism into which it is transfused without side effects.

The expression “non-toxic” means that the blood substitute does not cause any pathological disorder of an immune-reaction, allergic or nephrotoxic type.

The expression “has no pathogenic agent” refers to the absence of identified microorganisms or viruses.

The absence of pathological disorders indirectly implies the absence of pathogens.

The expression “keeps for at least 6 weeks at 4° C. without oxidation” means that the active site and in particular the iron present in the haem, which is involved in the oxygen bond remains in the form Fe²⁺ form (functional state). The oxidation of the active site is due to the passage of Fe²⁺→Fe³⁺ involving a possibility of binding oxygen.

The expression “transfusable into all blood types” refers to the absence of blood typing (ABO or rhesus system). This haemoglobin could be considered as a universal donor type haemoglobin.

The expression “has a sufficiently long residence time to ensure regeneration into natural haemoglobin of the organism into which it is transfused” refers to the presence of this haemoglobin in the blood system after at least 48 hours prior to transfusion. This time is long enough to enable an organism to resynthesise its own red blood corpuscles.

By way of illustration, within the framework of the transfusion of a human being, the time must advantageously be of the order of 48 hours.

The expression “eliminated by the organism into which it is transfused without side effects” means that this extracellular haemoglobin seems to be eliminated by natural means not giving rise to any particular pathological disorder.

In vertebrates, the life of a red blood corpuscle lasts approximately 120 days. The red corpuscle is then phagocyted (physiological haemolysis). The haemoglobin is then transformed into biliverdin and bilirubin which are eliminated by the bile.

None of the side effects likely to be encountered with products of the prior art, in particular oedemas, problems of immunogenicity and nephrotoxicity do not exist within the framework of the present invention.

According to an advantageous embodiment, in the blood substitute of the invention, the extracelluar haemoglobin comes from Annelids:

The classification to which reference is made when using the term Annelids is that described in Meglitsch P. A. (1972) (75).

According to an advantageous embodiment, in the blood substitute of the invention, the extracelluar haemoglobin comes from Arenicola marina.

In the extracellular haemoglobin of Arenicola marina, the number of free cysteines capable of binding to the NO and/or SNO groups is equal to 124.

Moreover, there are, in total, 156 intrachain disulphide bridges on the globin chains, as there is an intrachain bond (disulphide bond) on each globin chain and the molecule is made up of 156 globin-type chains (60).

With regard to intermolecular bonds, each twelfth of the molecule is made up of twelve globin-type chains associated as follows: 3 covalent trimers and 3 monomers. There are thus 52 intermolecular bonds between the globin chains.

### DESCRIPTION OF THE FIGURES

FIG. 1 represents the structure of the haemoglobin molecule.

The mammalian haemoglobin molecule is made up of four similar functional polypeptide chains in pairs (2 α-type globin chains and 2 β-type globin chains), each having the tertiary structure of a myoglobin molecule (11).

FIGS. 2A and 2B represent the model of hexagonal bilayer (HBL) haemoglobin of Arenicola marina.

FIG. 2A: Front view

Tn corresponds to the different trimers made up of globin-type chains b, c and d

FIG. 2B: detail of a twelfth

FIGS. 3A, 3B and 3C represent the haemoglobin of Arenicola marina viewed with transmission electron microscopy.

FIG. 3A: Overall view of a solution containing extracellular haemoglobin of Arenicola manna.

FIG. 3B: Front view of the molecule

FIG. 3C: Profile view

FIG. 4: Monitoring over 17 weeks of the weight of a group of 5 mice transfused with 1-2 g/% of haemoglobin of Arenicola, as described in the following examples.

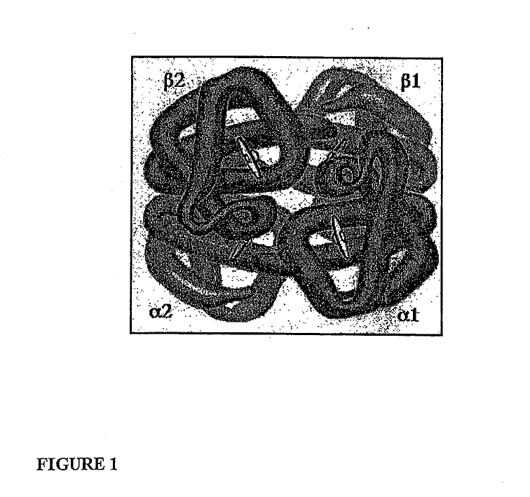

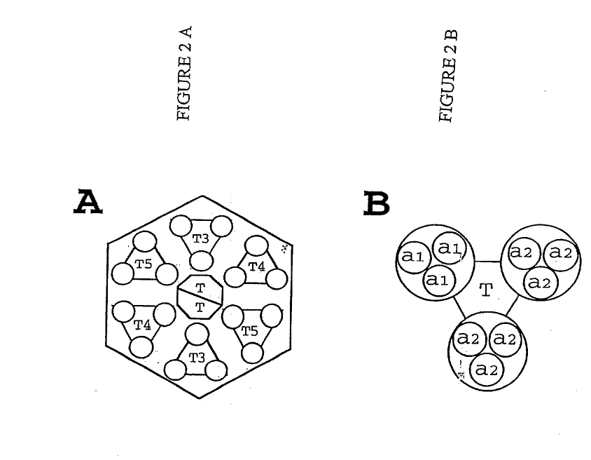

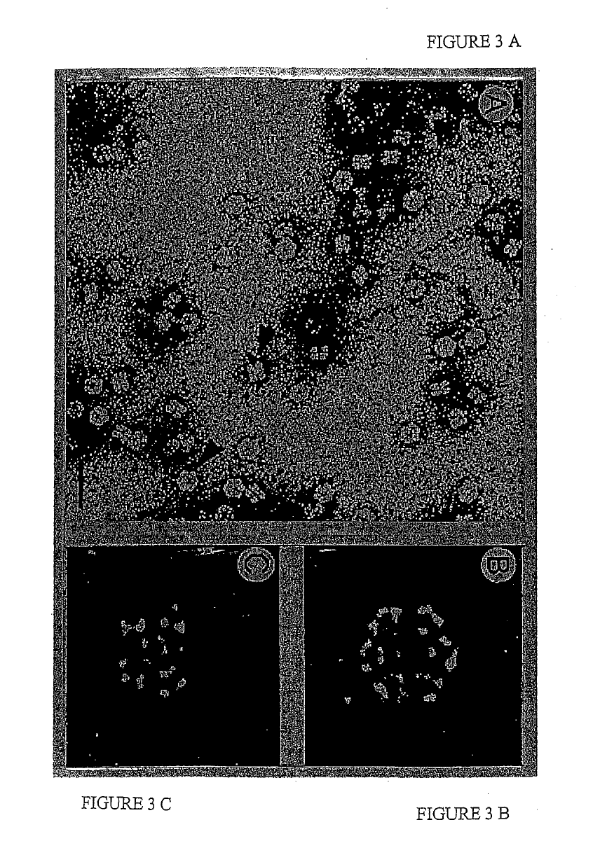

The x-axis corresponds to the weeks and the y-axis corresponds to the weight. The curve with the blank circles corresponds to the control mouse, that with the black circles to mouse no. 1, that with the white triangles to mouse no. 2, that with the black triangles to mouse no. 3, and that with the white squares to mouse no. 4.

Even after the exchange of blood, the mice continue to grow, the control mouse testifying to the animals' being in good condition.

After 9 weeks, two mice are retransfused with haemoglobin from Arenicola marina. Once again, no disorder is observed, attesting the lack of immunoreactivity or allergic response.

### EXAMPLES

Taking Haemoglobin Samples

The Arenicolae were harvested at low tide on the foreshore close to Saint-Pol de Leon, North Finisterre, France. The blood is taken from the ventral vessel after dissection on a bed of ice. The samples are taken using a glass micropipette connected to a mouth-suction system developed by Toulmond (1975) or 1 ml hypodermic syringes equipped with a 25 G×⅝″ needle. The samples are collected on ice. After cold centrifugation (15 000 g for 15 min at 4° C.) to eliminate any tissue debris, the supernatants are frozen at −20° C. or in liquid nitrogen, or immediately purified.

Purification of the Haemoglobins

Before purification, the thawed sample is centrifuged, at 5 000 g for 5 min at 4° C. After centrifugation, a small residue is generally present; this is eliminated.

Low pressure filtration-(FPLC, Pharmacia, LKB Biotechnology Inc.) of aliquots of 100 μl of supernatant is carried out using a Superose 6-C column (Pharmacia, separation range between 5.10³ and 5.10⁶ Da) or by simple chromatography using a 2.5×100 cm Sephacryl S-500 HR column (Amersham Pharmacia Biotech, separation range between 40 and 20 000 kDa). The samples are eluted with Riftia salinated buffer developed by Arp et al. (1987) and Fisher et al. (1988). The composition of this modified buffer is as follows, for one litre: 23.38 g NaCl (400 mM); 0.22 g KCl (2.95 mM); 7.88 g MgSO₄, 7H₂O (31.97 mM); 1.62 g CaCl₂, 2H₂O (11.02 mM) and HEPES (50 mM). The pH is adjusted to pH=7.0 by adding HCl. The rate used is generally 0.4 to 0.5 ml/min. The absorbance of the eluate is followed at two wave lengths: 280 nm (protein absorbance peak) and 414 nm (haemoglobin absorbance peak). The fractions containing the haem are concentrated using Centricon-100 (15 ml) tubes or using an agitation cell retaining the molecules with a weight above or equal to 10 000 Da. Two purification processes following the same protocol are necessary to obtain pure fractions.

Transfusion of ArHb into Mice

The aim of this experiment was to investigate the possibility of using extracellular haemoglobin of Arenicola marina (ArHb) as a blood substitute in a vertebrate model.

For this purpose, 30 adult male reproductive C57 BL/6J mice were used, whose mass was between 25 and 40 g. Four mice were used as a control. In general the blood volume of a mouse of this type is between 1.5 and 2 ml.

First the mice were anaesthetised with chloroform after being weighed and clearly identified.

Then 200 to 800 μl of blood were taken from the retro-orbital plexus and the blood of each mouse was centrifuged at low speed to recover the plasma (supernatant). This was kept carefully to be reinjected subsequently into the same mouse, with the ArHb. The previously purified ArHb is dissolved in the plasma at a concentration of 1.5 g %.

The mixture thus prepared was then injected into the caudal vein. In the case of the control mice, after a volume of blood was taken, the same volume of an isotonic saline solution containing their respective plasma was injected.

Finally, in the case of 5 mice, 10 μl of the mouse's blood before transfusion and 10 μl of blood after transfusion were kept to investigate the functional properties. In the case of five other mice, a 30 to 40 μl sample of blood was taken from the orbital plexus after 2 and 48 hours to analyse the functional properties and carry out spectrophometric studies allowing the possible identification of methemoglobin.

These mice were monitored for three months, observing more particularly their general behaviour and weight gain.

It was found that the mice transfused with the ArHb did not die and that their behaviour was similar to that of the control mice.

Analysis of the blood samples showed the following elements: i) the ArHb was still present after 48 hours preceding the transfusion; ii) no modification of the functional properties of the blood of the transfused mice; iii) no sign of the presence of methemoglobin.

Immunoreactivity

Two months after their first transfusion with ArHb, a new injection was carried out into the vascular system (2 mice) and intraperitoneally (2 mice). These 800 μl injections contained an isotonic saline solution in which the ArHb was dissolved (1-2 g/%).

No disorders were observed after recovery from anaesthesia, and these animals are still alive today.

This absence of immune response may be linked either to the size of this protein which would not allow activation of the immune system, or to the fact that after a few days the macrophages have totally eliminated these foreign proteins.

Functional Properties

P₅₀

The P₅₀ was measured using the hemox technique (1).

n₅₀

The n₅₀ was measured on the oxygen saturation curves of a respiratory pigment, obtained using the hemox technique.

The following table shows the measurements of P₅₀ (affinity) and n₅₀ (cooperativity) for Arenicola marina in comparison with the values of corresponding human haemoglobin. These measurements were obtained in vitro under the same conditions for the human haemoglobulin and that of Arenicola marina.

| | P₅₀ (mm Hg) | n₅₀ |
|---|---|---|
| Haemoglobin | 6.4 | 2.7 |
| of Arenicola marina | | |
| Human | 6.1 | 2.6 |
| haemoglobin | | |

As regards Arenicola marina, the value indicated is the average of three measurements.

These results show that the haemoglobin of Arenicola marina and human haemoglobulin (HbA) possess similar functional properties without any prior modification.

Extracellular Haemoglobins vis-à-vis NO/SNO

Nitrogen Monoxide (NO)

A blood vessel can be represented schematically by a cylinder made up of smooth muscular tissues on the outside, then a layer of endothelial cells in contact with the blood. This layer of endothelial cells plays an important role, as it is involved in the NO release processes. NO is the major factor controlling vascular tonus. When the concentration of NO in the blood is reduced, the vessels will be in a state of vasoconstriction and, conversely, an increase in NO will lead to vasodilation of the vessels (68). Nitrogen monoxide is also known as a neuromediator (69). It is also involved in other metabolism control mechanisms (70). The junctions between the endothelial cells allow tetrameric haemoglobin to cross this cell layer and be eliminated from the circulation. Consequently, as haemoglobin is capable of fixing nitrogen monoxide, it acts, on leaving the vessels, as a well for the NO, which gives rise not only to vessel-vasoconstriction phenomena, but also a number of neurological problems. At present, all the modified (bridged, polymerised or conjugated) haemoglobin solutions contain a small proportion of normal tetrameric haemoglobins crossing the endothelial cell layer. This problem is solved by using high molecular weight extracellular haemoglobins like those of Arenicola marina which are naturally polymerised and too large to cross the vessel wall.

Thionitrosyl Groups (SNO)

In addition to its role as a transporter of oxygen, the haemoglobin of vertebrates plays an important role in the transport of NO and SNO (71). Basically, it has been shown that oxyhaemoglobin had a greater affinity for SNO than deoxyhaemoglobin, that deoxyhaemoglobin had a greater affinity for NO than oxyhaemoglobin and that SNO was in particular produced in the lungs and that it had a major role in the control of vasoconstriction and vasodilatation of the vessels. It is interesting to note here that, with regard to the extracellular haemoglobin of Arenicola marina, it has been shown that only the haemoglobins belonging to marine worms colonising environments rich in hydrogen sulphide had the sites (presence of free cysteines on the globin-type chains) necessary to perform this function (58). This property was studied using the technique of Jia et al (71).

Extracellular Haemoglobins and SOD Activity

The red corpuscles contain a number of enzymes such as catalases and superoxide dismutases (SOD) which have an indispensable role in the deactivation of radical oxygen, a highly toxic compound. However, existing blood substitutes do not possess these activities as they are located outside the red corpuscles. An oxygenation deficit in the organism, caused by haemorrhagic shock or ischaemia, stimulates the production of hypoxanthine and activates xanthine oxidase. If this organism is then under oxygen, the xanthine oxidase will transform the hypoxanthine into superoxide which will give rise to radical oxygen. The enzyme superoxide dismutase will then have the role of transforming the radical oxygen into hydrogen peroxide, itself transformed into water by catalase. The first generations of blood substitutes lacked these enzymes, giving rise to a number of side effects. Although the new generations of products are attempting to overcome these problems, they have not been resolved, which gives a further advantage to the use of extracellular haemoglobins from Arenicola marina. This is because these molecules possess an intrinsic SOD activity which can be linked to the presence of structural chains (72,73).

The ArHb's SOD (superoxide dismutase) activity was measured, and values of the order of 10 U/mg of protein were found.

The SOD activity was studied using luminescence. This quantity determination is based on the competition between the SOD and an imidazolopyrazine for the superoxide anion. This anion, generated by the action of xanthine oxydase on hypoxanthine in the presence of oxygen, can react with imidazolopyrazine and produce light. In the presence of SOD, one part of the superoxide anions is consumed and the other oxidises imidazolopyrazine, of which there is an excess in the reaction medium, releasing the measured light. Thus the lower the SOD content in the sample, the higher the luminescence measured.

HPX(hypoxanthine)+XOD(xanthine oxidase)+O₂→uric acid+O₂⁻(superoxide ion)

2O₂⁻+SOD→H₂O₂

CL₉(coelenterazine)+O₂⁻→oxidated CL₉+hν

Transfusion of Haemoglobin of Arenicola into Mice

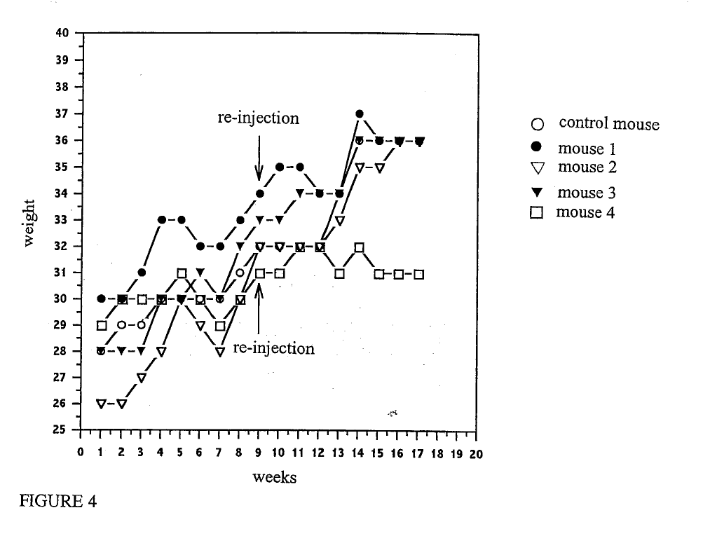

Approximately 50% of the volume of the blood is extracted and replaced by 1-2 g/% of haemoglobin of Arenicola marina. The haemoglobins of annelids are dissolved in the plasma of the animals or in a buffer before injection. The volume of substitute injected is essentially the same as the initial volume taken from the mouse. The most surprising observation is that there are no behavioural or physiopathological effects in these mice partially tranfused with haemoglobin of Arenicola marina (n=30), even 14 months later (FIG. 4).

Immunoreactivity

The mice retransfused 9 weeks after the initial transfusion with the haemoglobin of Arenicola marina show no allergic response and no deaths have occurred. In all these experiments, 200 μg of haemoglobin are transfused via the caudal vein into 2 experimental mice. After recovering from the anaesthesia, these mice behave normally. Two weeks after this transfusion (i.e. 12 weeks after the initial transfusion), the mice are retransfused with a solution of Arenicola marina haemoglobins by intraperitoneal injection, and again no allergy or pathological response could be observed (FIG. 4). It can therefore be concluded that the mechanisms of recognition by antigens resulting from the formation of antibodies are not activated by a protein of this size or that the macrophages eliminated this large protein with no apparent problem.

These new results lead to the conclusion that the size of these molecules can be a determining factor in allowing Arenicola marina haemoglobin to function non-toxically in vertebrates.

### REFERENCES

1. A. Toulmond et al., Biol. Bull. 179 366-373 (1990)

2. R. E. Hirsch, L. A. Jelicks, B. A. Wittenberg, D. K. Kaul, H. L. Shear and J. P. Harrington, Artificial Cells, Blood Substitutes & Immobilization Biotechnology 25 429-444 (1997).

10. N. B. Terwilliger, Molecular Structure of the extracellular heme proteins., Vol. 13, C. P. Mangum (Ed), 193-229, Springer-Verlag, Berlin (1992).

11. R. E. Dickerson and I. Geis, Hemoglobin: Structure, function, evolution, and pathology., Benjamin/Cummings, Menlo Park, Calif. (1983).

23. L. C. J. Clark and F. Gollan, Science 152 1755 (1966).

24. T. M. S. Chang, Hemoglobin Corpuscles, McGill University. (1957).

25. T. M. S. Chang, Science 146 524-525 (1964).

28. R. P. Geyer, R. G. Monroe and K. Taylor, Survival of rats totally perfused with a fluorocarbon-detergent preparation., J. C. Norman, J. Folkman, W. G. Hardisson, L. E. Rudolf and F. J. Veith (Eds), 85-96, Appleton Century Crofts, New York (1968).

29. J. G. Reiss, Vox Sang 61 225-239 (1991).

30. J. G. Reiss, Artificial Cells, Blood substitutes & Immobilization Biotechnology, An International Journal 22 945-1511 (1994).

31. T. H. Goodin, E. B. Grossbard, R. J. Kaufman, T. J. Richard, R. J. Kolata, J. S. AIIen and T. E. Layton, Crit Care Med 22 680-689 (1994).

32. H. Sloviter and T. Kamimoto, Nature 216 458 (1967).

33. R. Naito and Y. K., An improved perfluorodecalin emulsion. In Blood Substitutes and Plasma Expanders, G. A. Jamieson and T. J. Greenwalt (Eds), 81, Alan R. Liss Inc., New York (1978).

34. T. Mitsuno and R. Naito, Perfluorochemical Blood Substitutes., Excerpta Medica, Amsterdam (1979).

35. T. Mitsuno and H. Ohyanagi, Present status of clinical studies of fluosol-DA (20%) in Japan, K. K. Tremper (Ed), 169-184, Little Brown & Co, Boston (1985).

36. T. M. S. Chang, Blood substitutes: principales, methods, products and clinical trials, Vol. I, Karger, Basel, Switzerland (1997).

38. R. E. Dickerson and I. Geis, Hemoglobin: Structure, function, evolution, and pathology., Benjamin/Cummings, Menlo Park, Calif. (1983).

39. T. M. S. Chang, Biochem. Biophys. Res. Com. 44 1531-1533 (1971).

40. J. W. Payne, Biochem J. 135 866-873 (1973).

41. H. F. Bunn and J. H. Jandl, Trans Assoc Am Physicians 81 147 (1968).

42. K. Nho, D. Glower, S. Bredehoeft, H. Shankar, R. Shorr and A. Abuchowski, Biomaterials, Artificial Cells and Immobilization Biotechnology, An International Journal 20 511-524 (1992).

44. J. N. Lamy, B. N. Green, A. Toulmond, J. S. Wall, R. E. Weber and S. N. Vinogradov, Chem. Rev. 96 3113-3124 (1996).

45. J. Roche, M. Bessis and J. P. Thiery, Biochim. Biophys. Acta 41 182-184 (1960).

46. J. Roche, M. T. Bessis and J. P. Thiery, C. R. Soc. Biol. 154 73-80 (1960).

47. O. Levin, J. Mol. Biol. 6 95-101 (1963).

48. J. Roche, Electron microscope studies on high molecular weight erythrocruorins (invertebrate hemoglobins) and chlorocruorins of annelids., D. A. Munday (Ed), 62-80, Pergamon Press, Oxford (1965).

49. E. F. J. Van Bruggen and R. E. Weber, Biochim. Biophys. Acta 359 210-212 (1974).

50. O. H. Kapp and A. V. Crewe, Biochim. Biophys. Acta 789 294-301 (1984).

51. F. De Haas, F. Zal, V. You, F. H. Lallier, A. Toulmond and J. N. Lamy, J. Mol. Biol. 264 111-120 (1996).

52. F. De Haas, F. Zal, F. H. Lallier, A. Toulmond and J. N. Lamy, Proteins-structure fonction and genetics, 3 241-256 (1996).

53. F. De Haas, N. Boisset, J. C. Taveau, O. Lambert, S. N. Vinogradov and J. N. Lamy, Biophys. J. 70 1973-1984 (1996).

54. F. De Haas, J.-C. Taveau, N. Boisset, O. Lambert, S. N. Vinogradov and J. N. Lamy, J. Mol. Biol. 255 140-153 (1996).

58. F. Zal, Structure des hémoglobines extracellulaires d'Annélides et de Vestimentifères colonisant des milieux extrêmes. Les hémoglobines face aux sulfures., Thèse de l'Université Pierre-et-Marie-Curie (Paris VI) (1996).

59. F. Zal et al., Proteins: Structure, Function, and Genetics 29 562-574 (1997).

60. F. Zal, B. N. Green, F. H. Lallier, S. N. Vinogradov and A. Toulmond, Eur. J. Biochem. 243 85-92 (1997).

66. O. Ouchterlony and L. A. Nilsson, Immunochemistry, Vol. 10, D. M. Weir, L. A. Herzenberg and C. Blackwell (Eds), 32, Blackwell, Oxford (1986).

68. J. G. Umans and R. Levi, Annu. Rev. Physiol. 57 771-790 (1995).

69. J. Garthwaite and C. L. Boulton, Annu. Rev. Physiol. 57 683-706 (1995).

70. C. Nathan, FASEB J. 6 3051-3064 (1992).

71. L. Jia, C. Bonaventura, J. Bonaventura and J. S. Stamler, Nature 380 221-226 (1996).

72. S. I. Liochev, A. R. Ruchumov, S. N. Vinogradov and I. Fridovich, Archives of Biochemistry and Biophysics 330 281-284 (1996).

73. J. Blum and I. Fridovich, Archives of Biochemistry and Biophysics 228 617-620 (1984).

74. F. Zal et al., Proc. Natl. Acad. Sci, USA 95 8997-9002 (1998).

75. P. A. Meglitsch, Invertebrate Zoology, Oxford University Press, Oxford, 834 p. (1972)

1. Use, as a blood substitute, of an extracellular haemoglobin having a molecular weight of approximately 3 to approximately 4 million daltons, comprising chains of polymerised globins, containing free cysteines capable of binding to NO and/or SNO groups, and having a P₅₀ of approximately 6 to approximately 7 mm Hg at 37° C.

2. Blood substitute, in particular human blood substitute, comprising an extracellular haemoglobin having a molecular weight of approximately 3 to approximately 4 million daltons, comprising chains of polymerised globins, containing free cysteines capable of binding to NO and/or SNO groups, and having a P₅₀ of approximately 6 to approximately 7 mm Hg at 37° C.

3. Blood substitute according to claim 2, wherein the haemoglobin cooperativity coefficient is 2 to 3 (n₅₀).

4. Blood substitute according to claim 2 or 3, wherein the globin chains of extracellular haemoglobin are stabilised between themselves, by covalent bonds, in particular intermolecular disulphide bridges, and the globin chains are auto-stabilised by intramolecular disulphide bridges.

5. Blood substitute according to one of claims 2 to 4, wherein the extracellular haemoglobin comprises structural chains which confer a hexagonal structure on the haemoglobin.

6. Blood substitute according to one of claims 2 to 5, wherein the extracellular haemoglobin is capable of neutralising toxic compounds, such as hydrogen sulphide.

7. Blood substitute according to one of claims 2 to 6, wherein the extracellular haemoglobin does not necessitate any cofactor to release any oxygen possibly fixed onto the haemoglobin.

8. Blood substitute according to one of claims 2 to 7, wherein the extracellular haemoglobin possesses the following properties:
it is non-toxic
it has no pathogenic agent
it keeps for at least 6 weeks at 4° C. without oxidation
it is transfusable into all blood types
it has a sufficiently long residence time to ensure regeneration into natural haemoglobin of the organism into which it is transfused
it is eliminated by the organism into which it is transfused without side effects.

9. Blood substitute according to one of claims 2 to 8, wherein the extracellular haemoglobin comes from Annelids.

10. Blood substitute according to claim 9, wherein the extracellular haemoglobin comes from Arenicola marina.
